(19) United States
(12) Patent Application Publication
(10) Pub. No.: US 2020/0070590 A1
(43) Pub. Date: Mar. 5, 2020

(54) TREAD DETECTING DEVICE AND TREAD DETECTING MODULE

(71) Applicant: SILICON TOUCH TECHNOLOGY INC., HSINCHU (TW)

(72) Inventors: CHI-YUAN CHIN, HSINCHU (TW); KUEI-JYUN CHEN, HSINCHU (TW)

(21) Appl. No.: 16/532,853

(22) Filed: Aug. 6, 2019

(30) Foreign Application Priority Data
Aug. 31, 2018 (TW) 107130605

(51) Int. Cl. B60C 11/24; G01N 33/44; G01N 27/02; G01B 7/06

(52) U.S. Cl. CPC B60C 11/243; G01B 7/06; G01N 27/02; G01N 33/445

(57) ABSTRACT

A tread detecting device and a trend detecting module are provided. The tread detecting device is disposed at a first predetermined thickness of a tire. The tread detecting device includes a control module providing a voltage or a current, and a tread detection module including a first tread detecting layer. The first tread detecting layer is disposed at the first predetermined thickness of the tire. The control module is electrically connected to the tread detecting module. The control module detects a variation of an impedance value of the tread detecting module to determine whether the thickness of the tread of the tire is less than or equal to the first predetermined thickness.

### CROSS-REFERENCE TO RELATED PATENT APPLICATION

This application claims the benefit of priority to Taiwan Patent Application No. 107130605, filed on Aug. 31, 2018. The entire content of the above identified application is incorporated herein by reference.

Some references, which may include patents, patent applications and various publications, may be cited and discussed in the description of this disclosure. The citation and/or discussion of such references is provided merely to clarify the description of the present disclosure and is not an admission that any such reference is “prior art” to the disclosure described herein. All references cited and discussed in this specification are incorporated herein by reference in their entireties and to the same extent as if each reference was individually incorporated by reference.

### FIELD OF THE DISCLOSURE

The present disclosure relates to a tread detecting device, and more particularly to a tread detecting device disposed in a tire.

### BACKGROUND OF THE DISCLOSURE

In recent years, as driving safety has become more and more important, tread detection, tire pressure detection, eagle eye system or Advanced Driver Assistance Systems (ADAS) are key issues in the development of the industry today. Most tread detection devices mainly use the image capture systems. That is, camera devices are still used to detect current tread thickness, which causes a relatively higher cost and is unable to immediately report the current tire condition.

Therefore, it is an important subject in the industry to provide a tread detecting device combined with a tire.

### SUMMARY OF THE DISCLOSURE

In response to the above-referenced technical inadequacies, the present disclosure provides a tread detecting device. The tread detecting device is disposed at a first predetermined thickness of a tire. The tread detecting device includes a control module providing a voltage or a current, a tread detection module including a first tread detecting layer. The first tread detecting layer is disposed at the first predetermined thickness of the tire. The control module is electrically connected to the tread detecting module. The processing module detects a variation of an impedance value of the tread detecting module to determine whether the thickness of the tread of the tire is less than or equal to the first predetermined thickness.

In one aspect, the present disclosure provides a tread detecting module. The tread detecting module is disposed in a tire. The tread detecting module includes a base layer and a first tread detecting layer. The first tread detecting layer is disposed on the base layer. The base layer and the first tread detecting layer are flexible.

Therefore, the tread detecting device of the present disclosure can be disposed in the tire, so that a tread abrasion can be immediately reported, and the tread detecting device of the present disclosure can be implemented at any preset position of the tire. Therefore, the structure of the tread detecting device is adjusted based on the manufacturing process of the tire, without increasing the cost. In addition, the tread detecting device of the present disclosure can have a plurality of tread detecting layers for detecting different tread thicknesses, which can improve the flexibility of the design. Furthermore, the tread detecting device of the present disclosure can also be used as a detecting device for abnormal tire conditions, such as a detecting device for a puncture condition.

These and other aspects of the present disclosure will become apparent from the following description of the embodiment taken in conjunction with the following drawings and their captions, although variations and modifications therein may be affected without departing from the spirit and scope of the novel concepts of the disclosure.

### BRIEF DESCRIPTION OF THE DRAWINGS

The present disclosure will become more fully understood from the following detailed description and accompanying drawings.

FIG. 1 is a schematic diagram of a tread detecting device according to an embodiment of the present disclosure.

FIG. 2 is another schematic diagram of the tread detecting device according to the embodiment of the present disclosure.

FIG. 3A is a schematic diagram of a tread detecting module according to the embodiment of the present disclosure.

FIG. 3B and FIG. 3C are schematic diagrams showing the tread detecting module according to another embodiment of the present disclosure.

FIG. 4 is a schematic view showing the tread detecting device disposed on the inner side of the tire according to an embodiment of the present disclosure.

FIG. 5 is a schematic diagram of a vehicle system provided with a plurality of tread detecting devices according to an embodiment of the present disclosure.

FIG. 6 to FIG. 7 are schematic views showing the tread detecting device disposed on the surface of the tire according to an embodiment of the present disclosure.

FIG. 8 is a schematic view showing the tread detecting device disposed on the lateral side of the tire according to an embodiment of the present disclosure.

### DETAILED DESCRIPTION OF THE EXEMPLARY EMBODIMENTS

The present disclosure is more particularly described in the following examples that are intended as illustrative only since numerous modifications and variations therein will be apparent to those skilled in the art. Like numbers in the drawings indicate like components throughout the views. As used in the description herein and throughout the claims that follow, unless the context clearly dictates otherwise, the meaning of “a”, “an”, and “the” includes plural reference, and the meaning of “in” includes “in” and “on”. Titles or subtitles can be used herein for the convenience of a reader, which shall have no influence on the scope of the present disclosure.

The terms used herein generally have their ordinary meanings in the art. In the case of conflict, the present document, including any definitions given herein, will prevail. The same thing can be expressed in more than one way. Alternative language and synonyms can be used for any term(s) discussed herein, and no special significance is to be placed upon whether a term is elaborated or discussed herein. A recital of one or more synonyms does not exclude the use of other synonyms. The use of examples anywhere in this specification including examples of any terms is illustrative only, and in no way limits the scope and meaning of the present disclosure or of any exemplified term. Likewise, the present disclosure is not limited to various embodiments given herein. Numbering terms such as “first”, “second” or “third” can be used to describe various components, signals or the like, which are for distinguishing one component/signal from another one only, and are not intended to, nor should be construed to impose any substantive limitations on the components, signals or the like.”

### First Embodiment

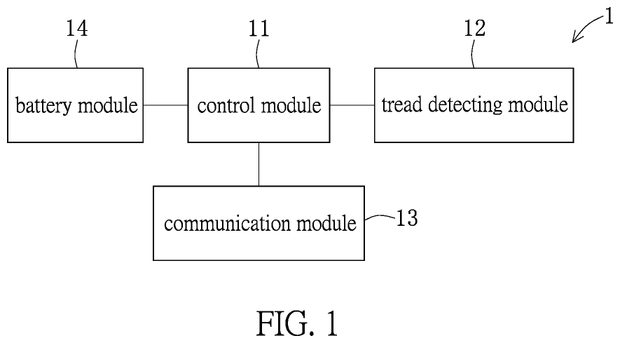

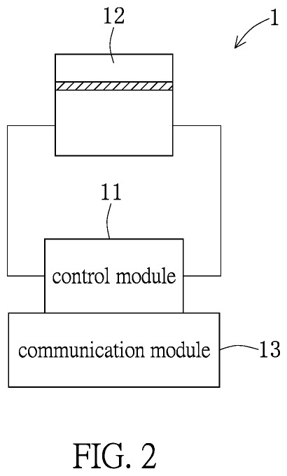

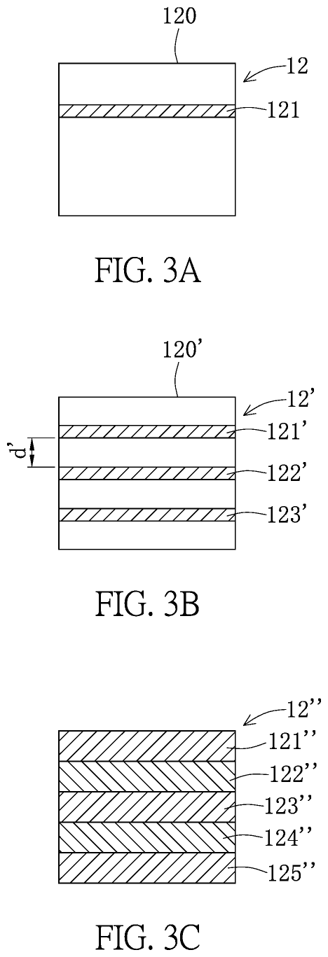

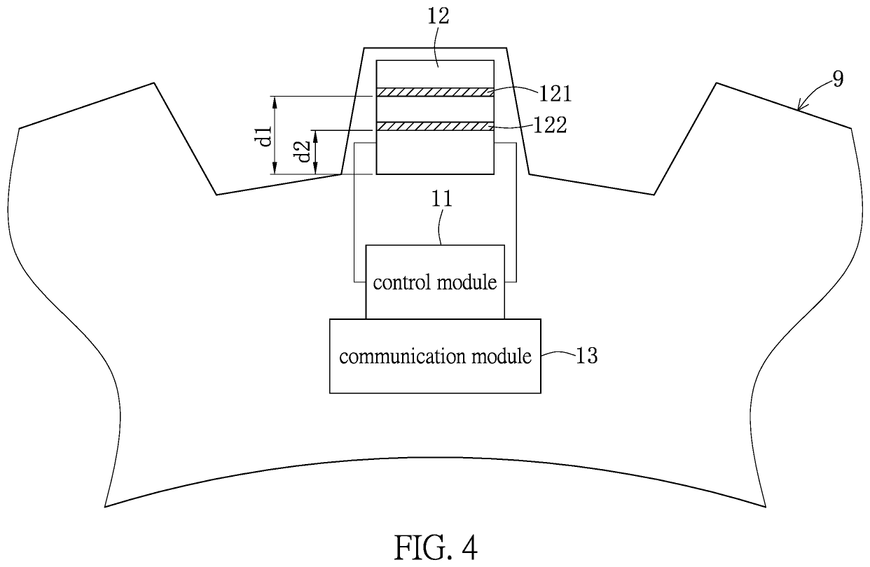

Referring to FIG. 1, FIG. 2 and FIG. 3A, FIG. 1 is a schematic diagram of a tread detecting device according to an embodiment of the present disclosure. FIG. 2 is another schematic diagram of the tread detecting device according to the embodiment of the present disclosure. FIG. 3A is a schematic diagram of a tread detecting module according to an embodiment of the present disclosure.

In the embodiment, a tread detecting device 1 includes a control module 11, a tread detecting module 12, and a communication module 13.

The control module 11 is electrically connected to the tread detecting module 12 and the communication module 13. In the embodiment, the control module 11 detects an impedance value of the tread detecting module 12. In other words, the control module 11 provides a voltage or a current through the tread detecting module 12 to detect the impedance value of the tread detecting module 12. In the embodiment, the tread detecting module 12 further includes a base layer 120 and a first tread detecting layer 121. The base layer 120 is an elastic body and may be rubber or other flexible material. In the present embodiment, the base layer 120 is the same material as the tire. The first tread detecting layer 121 is a metal plating film. In other words, when the tread detecting module 12 of the tread detecting device 1 is disposed in the tire, the tread detecting module 12 may also have a certain degree of deformation along with the deformation of the tire, which does not influence the structure of the tire. In the embodiment, the tread detecting module 12 is disposed at a first predetermined thickness. In other words, the tread detecting module 12 can be disposed at a predetermined position of different tread thickness based on actual requirements, for example, 5 mm or 6 mm.

In the embodiment, the tread detecting device 1 further includes a battery module 14 for supplying the control module 11 and the communication module 13. In the embodiment, the battery module 14 is a battery. In other embodiment, the battery module 14 can be an energy harvesting module. In other words, the energy supply module can collect a certain amount of energy by vibration, temperature, or electromagnetic, which can be designed based on actual requirements, and is not limited in the present disclosure. In the embodiment, the communication module 13 can be a wireless fidelity communication module (WiFi), a Bluetooth communication module (BlueTooth), a low-power Bluetooth communication module (BlueTooth Low Energy) or a Zigbee communication module, which is not limited in the present disclosure.

Referring to FIG. 3A, FIG. 3B, and FIG. 3C, FIG. 3B and FIG. 3C are schematic diagrams showing the tread detecting module according to another embodiment of the present disclosure.

As shown in FIG. 3A, a side view of the tread detecting module 12 according to the embodiment of the present disclosure, the first tread detecting layer 121 is disposed in the base layer 120. In the embodiment, the thickness of the first tread detecting layer 121 and the base layer 120 can be designed based on actual requirements, which is not limited in the present disclosure. In other embodiments, the first tread detecting layer 121 may be disposed on a surface of one side of the base layer 120. Therefore, the position and thickness of the first tread detecting layer can be designed based on actual requirements, which is not limited in the present disclosure. In the embodiment, the control module 11 can detect the impedance value of the first tread detecting layer 121 for determining whether the tire is worn to the first predetermined thickness. In other words, the control module 11 can detect a variation in the impedance value of the first tread detecting layer 121, and determine whether the tire is worn to the first predetermined thickness. In the embodiment, since the thickness of the first tread detecting layer 121 can be designed based on actual requirements, the control module 11 also has some conditions as following. If the first tread detecting layer 121 is originally designed to be very thin, for example, 0.01 mm, i.e., 10 um, the degree of tire abrasion varies during different driving cycles of the vehicle.

For example, when the control module 11 detects that the impedance value of the first tread detecting layer 121 of the tread detecting module 12 is 10 mΩ two days before, in the present the control module 11 detects that the impedance value of the first tread detecting layer 121 of the tread detecting module 12 is infinite. In other words, the control module 12 cannot detect the exact impedance value of the first tread detecting layer 121. In the embodiment, the control module 11 determines that the tire has worn to the first predetermined thickness. The control module 11 transmits a notification signal through the communication module 13. In other embodiments, the control module 11 can detect the variation of the impedance value of the first tread detecting layer 121. For example, when the control module 11 detects that the impedance value of the first tread detecting module 12 is 10 mΩ two days before, in the present the control module 11 detects that the impedance value of the first tread detecting layer 121 of the tread detecting module 12 is 50 mΩ. The control module 11 still determines that the tire has worn to a first predetermined thickness, and the control module 11 transmits a notification signal through the communication module 13.

That is, the control module 11 determines that the thickness of the tire tread is less than or equal to the first predetermined thickness. Therefore, the control module 11 transmits a notification signal through the communication module 13. In other words, as long as the control module 13 detects that the impedance value of the first tread detecting layer 121 changes, the control module 11 transmits a notification signal through communication module 13. In other embodiments, when the tire is pierced by a foreign object (for example, a nail or other non-metallic tip), the impedance value of the first tread detecting layer 121 changes. At this time, the control module 11 transmits a notification signal through the communication module 13 based on the impedance variation of the first tread detecting layer 121.

As shown in FIG. 3B, a tread detecting module 12′ includes a base layer 120′, a first tread detecting layer 121′, a second tread detecting layer 122′, and a third tread detecting layer 123′. The first tread detecting layer 121′, the second tread detecting layer 122′, and the third tread detecting layer 123′ are disposed in the base layer 120′. A first predetermined distance d′ is defined between the first tread detecting layer 121′ and the second tread detecting layer 122′. In the present embodiment, the distance between the second tread detecting layer 122′ and the third tread detecting layer 123′ is also defined as the first predetermined distance d′.

In the present embodiment, the first predetermined distance d′ is about 1 mm or 2 mm, which can be designed based on actual requirements, and is not limited in the present disclosure. In other embodiment, the first predetermined distance d′ and the second predetermined distance (not shown) may be set to different distances. In other words, when the first tread detecting layer 121′ is disposed at a first predetermined thickness of the tire, the second tread detecting layer 122′ and the third tread detecting layer 123′ may be respectively disposed at a second predetermined thickness (not shown) and a third predetermined thickness (not shown) of the inner side of the tire. In other words, the control module 11 of the tread detecting device 1 can detect the impedance values of the first tread detecting layer 121′, the second tread detecting layer 122′, and the third tread detecting layer 123′ to determine the current tread depth of the tire. In the embodiment, the user can set the notification for the thickness of the tread according to the running condition. For example, in normal driving conditions, the second tread detecting layer 122′ or the third tread detecting layer 123′ can be set as warning thresholds. However, if the tire is used in a large amount and is in a state of rapid wear, the first tread detecting layer 121′ can be set to transmit a notification signal, which can be designed based on actual requirements, and is not limited in the present disclosure.

As shown in FIG. 3C, the tread detecting module 12″ can include a plurality of tread detecting layers. That is, the tread detecting module 12″ includes a first tread detecting layer 121″, a first The second tread detecting layer 122″, a third tread detecting layer 123″, a fourth tread detecting layer 124″, and a fifth tread detecting layer 125″. In the embodiment, each tread detecting layer is designed in a laminated structure. In other words, the distance between the first tread detecting layer 121″ and the second tread detecting layer 122″ is zero. The distance between the second tread detecting layer 122″ and the third tread detecting layer 123″ is zero. The distance between the third tread detecting layer 123″ and the fourth tread detecting layer 124″ is zero. The distance between the fourth tread detecting layer 124″ and the fifth tread detecting layer 125″ is zero. In the embodiment, no insulating layer is provided between the first tread detecting layer 121″, the second tread detecting layer 122″, the third tread detecting layer 123″, the fourth tread detecting layer 124″, and the fifth tread detecting layer 125″. In other words, the first tread detecting layer 121″, the second tread detecting layer 122″, and the third tread detecting layer 123″, the fourth tread detecting layer 124″ and the fifth tread detecting layer 125″ are electrically conductive to each other. Therefore, the control module 11 can detect an overall impedance value of the first tread detecting layer 121″, the second tread detecting layer 122″, the third tread detecting layer 123″, and the fourth tread detecting layer 124″ and the fifth tread detecting layer 125″ for determining the thickness of the tread.

The user can set one or several thickness setting values for notifying, which is not limited by the present disclosure. In other embodiments, insulating layers may be disposed between the first tread detecting layer 121″, the second tread detecting layer 122″, the third tread detecting layer 123″, the fourth tread detecting layer 124″, and the fifth tread detecting layer 125″. The control module 11 is respectively connected to the first tread detecting layer 121″, the second tread detecting layer 122″, and the third tread detecting layer 123″, the fourth tread detecting layer 124″ and the fifth tread detecting layer 125″ to detect the impedance variation of the first tread detecting layer 121″ and the second tread detecting layer 122″, the third tread detecting layer 123″, the fourth tread detecting layer 124″, and the fifth tread detecting layer 125″, and the control module 11 transmits the notification signal, accordingly. In other embodiments, the first tread detecting layer 121″, the second tread detecting layer 122″, the third tread detecting layer 123″, and the fourth tread detecting layer 124″, and the fifth tread detecting layer 125″ can be integrally formed, such as a metal block. The control module 11 detects the impedance variation of the integrally formed layers including the first tread detecting layer 121″ and the second tread detecting layer 122″, the third tread detecting layer 123″, the fourth tread detecting layer 124″, and the fifth tread detecting layer 125″, and transmits a notification signal.

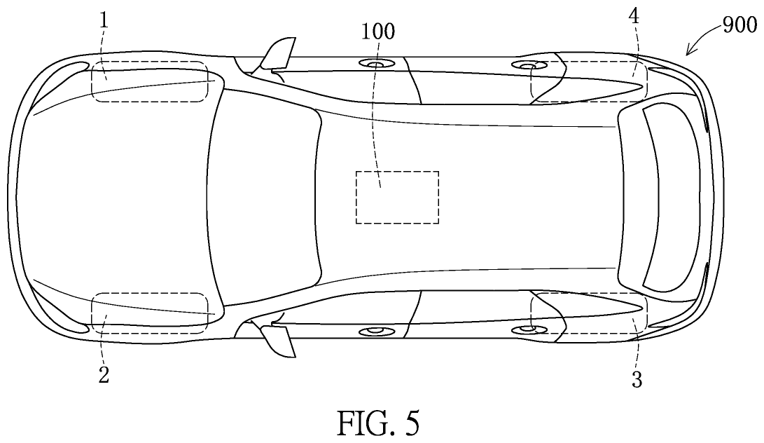

Referring to FIG. 4, FIG. 4 is a schematic view showing the tread detecting device disposed on the inner side of the tire according to an embodiment of the present disclosure.

In the embodiment, the tread detecting module 12 of the tread detecting device 1 is disposed at the tread protrusion of the tire 3, and the first tread detecting layer 121 is disposed at the first predetermined thickness d1 of the bottom side of the tread. In the embodiment, the tread detecting device 1 further includes a second tread detecting layer 122 disposed at a second predetermined thickness d2. In other words, in the embodiment, the tread detecting device 1 can detect whether the tread of the tire 3 is less than or equal to the first predetermined thickness d1 and whether it is less than or equal to the second predetermined thickness d2. In other embodiments, the location of the tread detecting module can be adjusted based on actual requirements, which is not limited in the present disclosure. In addition, in the embodiment, the control module 11 and the communication module 13 are disposed in the tire body. In other embodiments, the control module 11 and the communication module 1 may be disposed on the inner side of the tire, and the arrangement and position thereof are not limited in the present disclosure.

### Second Embodiment

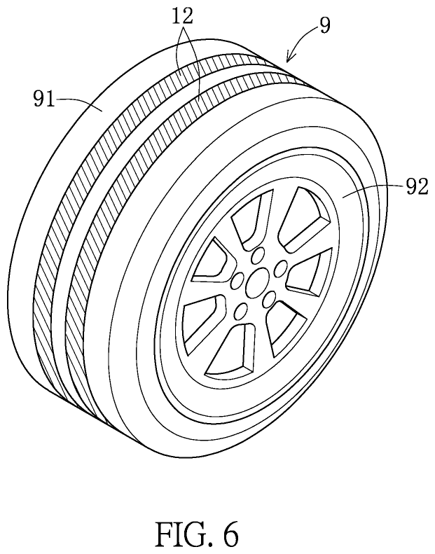

Referring to FIG. 5, FIG. 5 is a schematic diagram of a vehicle system provided with a plurality of tread detecting devices according to an embodiment of the present disclosure.

In the embodiment, a vehicle system 900 includes four tires, which can respectively be provided with a tread detecting device 1, a tread detecting device 2, a tread detecting device 3, and a tread detecting device 4. The respective control modules of the tread detecting device 1, the tread detecting device 2, the tread detecting device 3, and the tread detecting device 4 can detect the respective tread detecting modules to notify a vehicle computer device 100 of the vehicle system 900. In other embodiments, the number and position of the tread detecting device provided by the vehicle system 900 can be adjusted based on actual requirements, and are not limited in the present disclosure.

### Third Embodiment

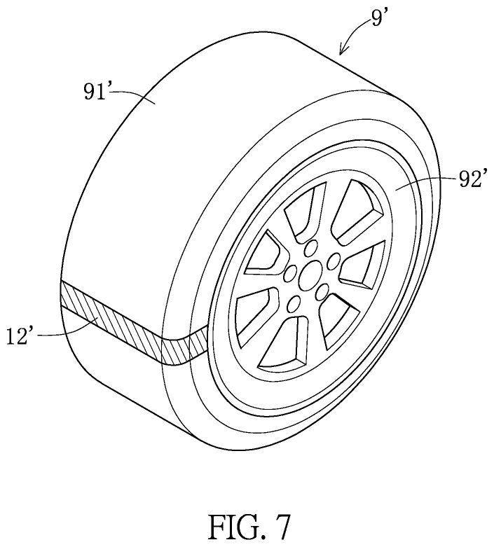

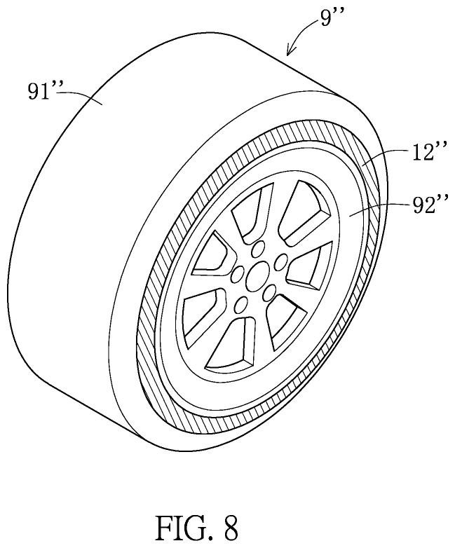

Referring to FIG. 6, FIG. 7 and FIG. 8, FIG. 6 to FIG. 7 are schematic views showing the tread detecting device disposed on the surface of the tire according to an embodiment of the present disclosure, and FIG. 8 is a schematic view showing the tread detecting device disposed on the lateral side of the tire according to an embodiment of the present disclosure.

In the present embodiment, a tire 9 includes a tread surface 91 and a lateral surface 92. Generally, the tread pattern is disposed on the tread surface 91, and most of the lateral surface 92 is provided with only a decorative pattern. Although the abnormal condition of the tire 9 mainly occurs in the tread surface 91, a small amount of acute subject puncture the lateral surface 92 of the tire 9. Therefore, in the present embodiment, the tread detection modules 12, 12′, 12″ can be defined as shown in FIG. 6, FIG. 7 and FIG. 8.

As shown in FIG. 6 and FIG. 7, the tread detecting module 12 can be disposed longitudinally on the tread surface 91 of the tire 9. Alternatively, the tread detecting module 12′ in FIG. 7 may be laterally disposed in a portion of the tread surface 91′ of the tire 9′ and a portion of the side surface 92′. In other embodiments, the tire tread may be a discontinuous protruding block (for example, a tire tread used in a tire for an agricultural vehicle or an industrial vehicle), and the tread detecting module 12 of the embodiment of the present disclosure may be selectively disposed at the tread protrusion, and arrangement position thereof is not limited in the present disclosure.

Referring to FIG. 8, the tread detecting device 12″ of the embodiment of the present disclosure may also be disposed on the side surface 92″ of the tire 9″ for detecting whether the side surface 92″ of the tire is penetrated by an acute subject. The arrangement of the tread detecting device 12″ is not limited in the present disclosure.

In conclusion, the tread detecting device of the present disclosure can be disposed in the tire, so that a tread abrasion can be immediately reported, and the tread detecting device of the present disclosure can be implemented at any preset position of the tire. Therefore, the structure of the tread detecting device is adjusted based on the process of the tire, which is without increasing the cost. In addition, the tread detecting device of the present disclosure can also have a plurality of tread detecting layers for detecting different tread thicknesses, which can improve the flexibility of the design. Furthermore, the tread detecting device of the present disclosure can also be used as a detecting device for abnormal tire conditions, such as a detecting device for a puncture condition.

The foregoing description of the exemplary embodiments of the disclosure has been presented only for the purposes of illustration and description and is not intended to be exhaustive or to limit the disclosure to the precise forms disclosed. Many modifications and variations are possible in light of the above teaching.

The embodiments were chosen and described in order to explain the principles of the disclosure and their practical application so as to enable others skilled in the art to utilize the disclosure and various embodiments and with various modifications as are suited to the particular use contemplated. Alternative embodiments will become apparent to those skilled in the art to which the present disclosure pertains without departing from its spirit and scope.

What is claimed is:

1. A tread detecting device disposed at a first predetermined thickness of a tire, the tread detecting device comprising:
a control module providing a voltage or a current; and
a tread detection module including a first tread detecting layer disposed at the first predetermined thickness of the tire;
wherein the control module is electrically connected to the tread detecting module;
wherein the processing module detects a variation of an impedance value of the tread detecting module to determine whether the thickness of the tread of the tire is less than or equal to the first predetermined thickness.

2. The tread detecting device of claim 1, further comprising: a communication module, wherein the control module transmits a tread determination result to a vehicle computer through the communication module.

3. The tread detecting device of claim 1, wherein the tread detecting module further includes a second tread detecting layer disposed at a second predetermined thickness of the tire.

4. The tread detecting device of claim 3, wherein a predetermined distance is defined between the first predetermined thickness of the tire and the second predetermined thickness of the tire.

5. The tread detecting device of claim 4, wherein the predetermined distance is zero.

6. The tread detecting device of claim 4, wherein the first tread detecting layer and the second tread detecting layer are integrally formed.

7. The tread detecting device of claim 1, further comprising:
a battery module providing the control module and the communication module with a driving energy.

8. The tread detecting device of claim 1, wherein the first tread detecting layer is disposed at a tread side of the tire.

9. The tread detecting device of claim 1, wherein the first tread detecting layer is disposed at a lateral side of the tire.

10. The tread detecting device of claim 1, wherein the first tread detecting layer is disposed on a lateral side of the tire and a tread side of the tire.

11. A tread detecting module disposed in a tire, the tread detecting module comprising:
a base layer; and
a first tread detecting layer;
wherein the first tread detecting layer is disposed on the base layer;
wherein, the base layer and the first tread detecting layer are flexible.
